(19) United States
(12) Patent Application Publication
(10) Pub. No.: US 2006/0122632 A1
(43) Pub. Date: Jun. 8, 2006

(54) Disposable device for multi-ligation of bleeding esophageal varices

(76) Inventors: Ali Mohamed Ali Hassanien, Alexandria (EG); Al-Taher Ali Sulaiman Murad, Dubal (UA)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/540,955

(22) PCT Filed: Dec. 27, 2003
(86) PCT No.: PCT/EG03/00014

§ 371 Date: Jun. 27, 2005

(30) Foreign Application Priority Data
Dec. 28, 2002 (EG) 2002121401

(51) Int. Cl. A61B 17/10

(52) U.S. Cl. 606/139

(57) ABSTRACT

The present invention relates to device for treating bleeding esophagus. The said device consists of three parts of cylinders. The first part characterized by its interior ⅔ is in the form of toothed rows with equal dimensions and parallel to each other along its circular surface. Its posterior ⅓ is in the form of two cylinders inside each other and connected together at its junction with the anterior ⅔ part leaving a specific space in between. The second part represent part of cylinder with three toothed arms situated vertically on it and similar in shape and dimensions. The third part consists of two cylinders inside each other and connected together on one side and free on the other side to be compatible with the other parts.

### TECHNICAL FIELD

This invention relates to disposable device for ligation of bleeding Esophageal varices. In particular the invention directed to a new disposable device for the ligation of bleeding Esophageal varices characterized in that it has a new mechanism and can be used for multi-ligation of the Esophageal varices.

### BACKGROUND OF THE INVENTION

Bleeding Esophageal varices is a severe complication of various liver diseases as a result of the high venous pressure on the esophageal region, The main diseases that lead to the Bleeding Esophageal varices includes

1—Liver parasites such as Bilharzias

2—Various kinds of Hepatitis.

3—Liver fibrosis of various origin (e.g. from drugs, alcohol liver toxicity etc.

Due to the high venous pressure on the esophageal region, hemorrhoids start to develop and immediately bleeding occurs. Where bleeding occurs, there is a need to achieve hemostasis in bleeding varices and minimize recurrence of this bleeding which may lead eventually to death. Presently, these Bleeding Esophageal varices are treated by any of the following methods

1—by pass surgery to redirect the main veins and reduce pressure in the esophageal region.

2—Injection sclerotherapy using endoscope.

3—band ligation utilizes an endoscope.

Prior Art:

Currently, therapeutic techniques includes injection sclerotherapy and band ligation. These techniques are utilized for the immediate and long term control of hemorrhaging associated with esophageal varices.

In sclerotherapy, a solution, such as sodium morrhuate or ethanolamine is injected submucosally, utilizing endoscope, into the tissue around the varicose vein in the esophagus to cause inflammation and scarring to close off the vein and reduce the likelihood of bleeding.

Disadvantages of these methods include the following:

1—It needs experienced surgeon to handle the injection to avoid injected the material in non-target regions.

2—Some of the bleeding veins can not be handled by this process.

3—repeated injection may create ulcerations or fibrosis which can lead to esophageal strictures

Ligation is effective method to treat this case using endoscope. The esophageal varices are ensnared with elastic bands to eradicate the varices.

Presently, there is a device for effecting this kind of ligation, this device is working through a mechanism that includes pulling the varices to the unit and release the rubber band by pulling a string

The use of this device is very critical since it may cause a damage to the endoscope itself and releasing of one rubber band only depend on the pulling forces if it too strong two or more rubber bands may be released.

Other ligation devices are known but the can be used to ligate only one varices at a time and then has to be taken out and reloaded which is inconvenient for both of the patient and the surgeon.

In U.S. Pat. No. 5,707,355, Zimmon; a method and apparatus for treating esophageal varices and mucosal neoplasm. In this method, a balloon tamponade device is utilized to deliver a dressing containing a sclerosant agent to bleeding esophageal.

U.S. Pat. No. 5,400,770, Nakao et at, disclosed a surgical device for use with endoscope to perform gastroesophageal hemostasis operations. The device comprises an insertion member, inflatable balloon and attachment component.

U.S. Pat. No. 6,139,527, Laufer et at, disclose a method and apparatus for treating hemorrhoids wherein a catheter delivers an electrode within a vein for a minimally invasive treatment of hemorroids using RF energy.

Yet a need exists in the art to treat the bleeding veins which give rise to esophageal varices and reduce venous pressure on the esophageal region from the portal vein system without the attendant risks of invasive surgery. Further need exists to provide a less invasive procedure which can treat multiple venous sites quickly and easily. There is still a need to achieve hemostasis in bleeding varices and minimize recurrence of bleeding.

### BRIEF DESCRIPTION OF THE DRAWINGS

FIG. 1 is schematic perspective view showing the new device of the invention.

FIG. 2. schematic perspective view showing the threads.

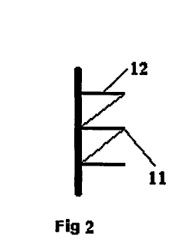

FIG. 3 cross sectional view showing the components of the cylinder.

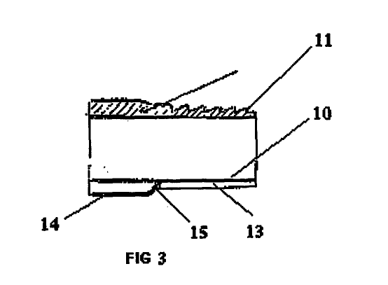

FIG. 4. Cross sectional taken along the line A-A of FIG. 1.

FIG. 5. schematic end view of the cylinder showing the position of the three arms.

FIG. 6. Cross sectional view showing the position of the pressure tube.

### DETAILED DESCRIPTION OF THE INVENTION

The present invention relates to device for multi-ligation of bleeding Esophageal varices. The new device will be better explained with reference to the accompanying drawings.

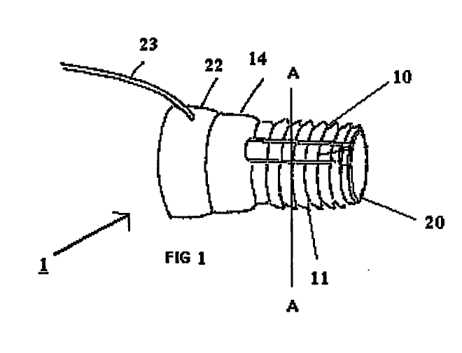

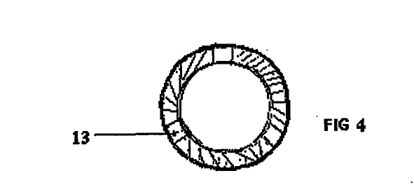

As illustrated in FIG. 1, the device (1) consists of three main parts. The first part is a cylinder (10) characterized in that the first two thirds of this cylinder is threaded. Threads (11) are circumscription and as shown in FIG. (2) it has a right angled triangle cross section. wherein the altitude (12) of the said triangle upwardly positioned. Distances and dimensions of all threads are identical. Cutting these threads longitudinal three identical equally spaced grooves in the form of passageways allowing tongues to pass through as better shown in FIG. 4 which is cross section taken along line A-A of FIG. 1

The last third of the cylinder (10) is not threaded and covered by another cylinder (14). Both cylinder having equal length, however, cylinder (14) is bigger in diameter. Cylinders 10 and 14 are integrated completely except for the position of the three grooves (13). Fig three represent a cross section of the two unified cylinders (10) and (14)

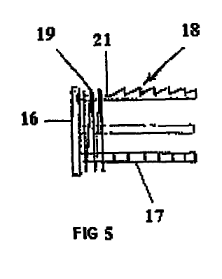

The second part is shown in FIG. 5 and consists of a base cylinder (16) having three arms (17). The arms (17) having threads (18) which are identical in all respects with threads (11) of the first part of the device. And having unthreaded part (21) connecting the threaded arms with the base cylinder. Dimensions of the arms (17) adopted to fit in the grooves (13) easily and to form altogether homogeneous unit with the first part. The base cylinder (16) is introduced in the space created by the last third of cylinder (10) and cylinder (14) so that arms can be inserted in the grooves and base cylinder moves upwardly in the said space and the arms (17) emerge and appeared on the surface of the threaded part of cylinder (1) and integrated therewith so that when arm (17) reach the top part of the device (20) the threads of the cylinder (10) are completed by the threaded arms (17). Within the unthreaded part on the arm (17), there is a special spring (19) which is used to trigger the arms upwardly and return it back after triggering.

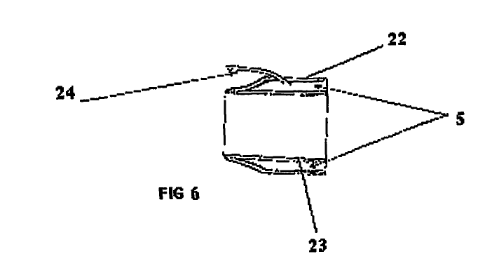

FIG. 6 represents the third part of the device which consists of two uni-center cylinders (22) and (23) one having bigger diameter (23) than the other which create a space (5) in between. There is a micro tube (24) connected to the said space from one side and connected to pressure source (not shown in the drawing) from the other side.

As mentioned above the said device can be easily used to ligate bleeding Esophageal varices using rubber bands loaded inside the device. Number of bands loaded into device canb vary from 4 up to 10, preferably from 4 to 8 and most preferable 6 rubber bands.

The practice of the invention is illustrated in the following non-limiting example.

Exemplary Procedure:

The device in this example is loaded with 6 rubber bands so that the first rubber band is immediately in front of the three arms.

The unit is connected with the endoscope and inserted into the patient from the mouth up till Esophagea. The surgeon determoine the bleeding varices which is withdrawn by the endoscope. Using a small air pressure (e.g. air pressure created by a syringe) the surgeon trigger the first rubber band upon using the pressure. Once pressure is used the three arms pushes the first rubber band to ligate the varices already withdrawn and in the mean time the spring return the arms back to the original position releasing a new rubber band to replace the one already used.

We claims:

1- New disposable device for ligation of bleeding Esophageal varices charctreized in that it consists of three main cylindrical parts.

2- New disposable device for ligation of bleeding Esophageal varices according to claim 1 wherein the first part is a cylinder characterized in that the first two thirds of this cylinder is threaded. The last third of the cylinder is not threaded and covered by another cylinder. Both cylinders having equal length, however, one of them is bigger in diameter. Cylinders are integrated completely except for the position of the three grooves

3- New disposable device for ligation of bleeding Esophageal varices according to claim 2 wherein Threads are circumscription and have a right angled triangle cross section wherein the altitude of the said triangle upwardly positioned. Distances and dimensions of all threads are identical.

4- New disposable device for ligation of bleeding Esophageal varices according to claim 2 wherein threads are cut longitudinal by three identical equally spaced grooves in the form of passageways allowing tongues to pass through.

5- New disposable device for ligation of bleeding Esophageal varices according to claim 1 wherein the second part consists of a base cylinder having three arms emerging therefrom. The arms having threads which are identical in all respects with threads of the first part of the device, and having unthreaded part connecting the threaded arms with the base cylinder. Within the unthreaded part of the arm, there is a special spring which is used to trigger the arms upwardly and return it back after triggering.

6- New disposable device for ligation of bleeding Esophageal varices according to claim 5 wherein threads are identical in all respects with threads of the first part of the device.

7- New disposable device for ligation of bleeding Esophageal varices according to claim 1 the third part of the device consists of two uni-center cylinders one having bigger diameter than the other creating a space in between. There is a micro long tube connected to the said space from one side and connected to pressure source from the other side.

8- New disposable device for ligation of bleeding Esophageal varices wherein it can be loaded with a number of rubber bands amounting to 6 or more.

9- New disposable device for ligation of bleeding Esophageal varices characterized in that one rubber band only can be rleased each time the device is triggered.

10- New disposable device for ligation of bleeding Esophageal varices wherein the mechanism of releasing the rubber band depend on a slight pressure created either by air or distilled water.

11- New disposable device for ligation of bleeding Esophageal varices according to claim 10 wherein the pressure source used can be medical syreinge.

12- New disposable device for ligation of bleeding Esophageal varices substantially as described hereinbefore with refreence to accompanying drawings.
